(19) United States
(12) Patent Application Publication
(10) Pub. No.: US 2012/0123442 A1
(43) Pub. Date: May 17, 2012

(54) ADHESIVE PATCH WITH ENZYME

(75) Inventors: Sanne Hartelius Larsen, Alleroed (DK); Leonardo Malcovati, Lyngby (DK)

(73) Assignee: Coloplast A/S, Humlebaek (DK)

(21) Appl. No.: 13/383,499

(22) PCT Filed: Jul. 14, 2010
(86) PCT No.: PCT/DK10/50188

§ 371 Date: Jan. 11, 2012

(30) Foreign Application Priority Data
Jul. 17, 2009 (DK) PA 2009 70070

(51) Int. Cl. A61B 17/50

(52) U.S. Cl. 606/131

(57) ABSTRACT

An adhesive patch for the removal or thinning of keratinaceous tissue comprising a backing layer covered on one side by an adhesive layer and an enzyme material comprising salmon zonase. The enzyme material selectively attacks the keratin, but not living cells.

### BACKGROUND OF THE INVENTION

1. Field of the Invention

The invention relates to an adhesive patch for application to callous skin and corns and to a method of removing or thinning keratinaceous tissue.

Hyperkeratosis, or thickening of the stratum corneum, is a widespread condition that can either be a symptom of a certain pathological condition, such as psoriasis, arsenic poisoning, certain forms of dermatitis, ichthyosis or it may simply be caused by excessive wear of an area of the skin, callus. In both cases, the problem is often local and may therefore be treated with an occlusive patch comprising an active chemical to aid the degradation of keratin. Other common methods for managing hyperkeratinized tissue are the application of acids in lotion or cream carriers, moisturizing creams or lotions or mechanical scraping. However, acids and scraping are disadvantageous because they tend to irritate normal epidermis and moisturizing creams are not effective in reducing hyperkeratotic tissue density.

Most common patches for hyperkeratosis simply rely on skin moisture to macerate the keratin layer by preventing moisture evaporation. Other solutions include active chemicals such as salicylic or lactic acid, combinations of the two or other compounds, such as α-hydroxy or β-hydroxy acids. Non-occlusive solutions comprise the usage of stronger acids, such as trichloroacetic acid, or surgical removal.

2. Description of the Related Art

Different ways of treating callous skin and corns are known.

Proteolytic enzymes may be used for degrading the hyperkeratotic tissue. U.S. Pat. No. 5,213,978 and U.S. Pat. No. 5,395,613 disclose the use of proteases derived from the bacterium Micrococcus sedentarius, and U.S. Pat. No. 6,858,215 disclose the use of subtilisin and fungal proteases for callous tissue.

All of the previous solutions suffer from two complementary setbacks: either they are only weakly effective or they are likely to cause damage to non-keratinaceous tissue. Patches that only rely on humidity typically take several weeks to remove the most common forms of callus and corns, whereas those that rely on α-hydroxy or β-hydroxy acids may cause irritation to healthy skin. Bacterial proteases, as well as those derived from plants, degrade a variety of proteins and are also liable to cause damage to healthy tissue. Furthermore, proteases lacking selectivity usually cause autolysis, thereby compromising their own long-term stability.

Thus, there is still a need for a patch that is capable of degrading keratinaceous tissue without damaging the healthy tissue.

### DETAILED DESCRIPTION OF THE PRESENT INVENTION

The invention relates to an adhesive patch for the removal or thinning of keratinaceous tissue, the patch comprising a first skin-facing surface and a second non-skin-facing surface, the patch comprises an adhesive layer, a backing layer and an enzyme material comprising salmon zonase.

Instead of chemicals that indiscriminately attack all human tissues, the patch comprises an enzyme material that selectively degrades only keratin and keratinaceous materials. The enzyme material is concentrated and protected from wash-out and degradation by the backing layer. The nature of the patch makes it easier to place the active enzyme material on the area that needs to be treated, which greatly increases its efficacy and cost-effectiveness.

The enzyme material may be produced by fish of the genus Salmo, the enzyme material comprises one or more proteases capable of degrading human keratin or keratinaceous material.

This solution offers a faster and, at the same time, safer way of treating hyperkeratosis.

Proteases define a subclass of enzymes with the ability to cleave proteins into smaller parts. The enzyme material comprising zonase softens and thins hyperkeratotic formations on the skin, including calluses, corns, dry scaling/flaking skin and keratoses without harming the surrounding living tissue by selective lysing of hyperkeratotic tissue. The enzymes accomplish this by lysing only the 10-end peptide bonds of keratin protein, the dominant protein component of hyperkeratotic skin.

Zonase is a fish-derived protease, displaying a high selectivity towards the degradation of human skin keratin, while leaving other proteins unaltered. Zonase is produced from natural sources, i.e. the Atlantic salmon, Salmo salar. Zonase is a non-self-degrading endoprotease which in nature serves as a hatching enzyme.

Zonase is an endoprotease, but unlike most digestive enzymes which degrade water-soluble polypeptides to small oligopeptides or amino acids, zonase performs strategic cuts in the macromolecular structure of the types of keratin involved in hyperkeratosis. Zonase is a serine-protease, cleaving proteins at the carboxyl-terminal side of arginine and lysine residues. However, zonase is not a substrate for its own proteolysis, rendering the enzyme reasonably stable in aqueous or dry state, even in the absence of stabilizing agents. The enzyme is described in detail in U.S. Pat. No. 6,592,866.

Zonase can degrade keratin, thereby enhancing removal of dry and dead cells from the skin surface, but the enzyme does not attack living human cells, i.e. living tissue. The catalytic specificity of the zonase is unique as the enzyme shows no toxic effects on living human cells in culture. The enzyme material is water soluble and has an optimum pH for protease activity at about 7.2.

By using the zonase enzyme for removal or thinning of keratinaceous tissue, the removal of dead skin cells is enhanced, without any harmful impact on living tissue. Keratin in human skin is only exposed in dead cells. The enzyme will selectively attack and degrade the dead cells, thereby thinning and over time totally removing the callous tissue to which it is applied.

The invention also relates to a method for removing or thinning hyperkeratotic mammalian tissue comprising the steps of: obtaining an adhesive patch comprising a backing layer covered on one side by an adhesive layer, the patch comprising enzyme material comprising a salmon zonase, and applying said patch to said hyperkeratotic tissue. The patch may be especially suitable for application to corns and callous skin.

The combination of active enzyme materials with adhesive patches can be technically complex. The enzyme material may be distributed in the adhesive of the patch or it may be located on the skin-facing surface of the patch.

The enzyme material may be located in one or more separate portions constituting at least a part of the skin-facing surface of the patch.

The enzyme containing portion may be partly embedded in the adhesive layer or the enzyme containing portion may in the form of a layer overlying a part of the second surface of the adhesive layer. Such layer may be in the form of a sheet or disc.

The enzyme material may be incorporated in a pharmaceutically or cosmetically acceptable carrier. The carrier may be a water-based carrier or an oil-based carrier. The carrier may comprise petrolatum, poly(ethylene glycol) or cocoa butter or it may comprise a hydrogel.

The carrier comprising the enzyme material may be in the form of one or more portions, for example in the form of discs being placed on the skin-contacting surface of the patch, or it may be located in indentations in the adhesive of the patch.

The carrier may further comprise chemicals to regulate the pH and enzymatic activity of the enzyme or to help its action.

A disc of the carrier material including the enzyme material may be an integrated part of the patch or the disc and the patch may be separate units being combined immediately before use.

The patch comprises a backing layer located on the second surface of the patch and an adhesive layer on the first skin-facing surface of the patch, the adhesive layer facilitating an adhesive surface for attachment of the patch to the skin. A portion comprising the carrier with the enzyme material may be located in one or more indentations in the adhesive layer or on the skin-facing surface thereof. The carrier comprising the enzyme material and the adhesive layer may be separated by a barrier layer capable of preventing migration from one component to another.

The adhesive may be any suitable skin compatible adhesive. The adhesive may contain hydrocolloids.

For use as topical application of an enzyme material to a corn, wart or callous skin, the skin contacting surface of the enzyme containing portion may be designed to substantially correspond in size to corn, wart or callous. The patch may protect the corn or wart while applied, both by the relatively impermeable backing layer and it may also act as a cushion if the adhesive layer has a certain thickness. After use, the patch can easily be peeled off and discarded.

The sizing of the enzyme carrying portion facilitates precise delivery of the enzyme only to the area to be treated. However, if the area of the enzyme containing material is larger than the corn thereby covering healthy living skin, the zonase will, contrary to other proteolytic enzymes, not attack the living skin cells and thereby cause damage.

Enzymes may be expensive and having the enzyme located only on the spot where it is needed may reduce the cost price significantly.

The patch renders it possible to treat a skin surface over a long period. The patch keeps the enzyme in place and protects it from being washed or worn out before the treatment is completed. The patch may be worn for several days allowing the enzyme to work undisturbed.

It is known to treat skin conditions like dry skin and psoriasis with Zona skin cream. The cream contains the enzyme zonase. However, the cream has to be applied at least twice a day for some time, and not washed off in between. Furthermore it is difficult to apply a cream locally to, for example, a corn.

The backing layer of the patch, according to the invention, may be any layer, such as a polyurethane film, foam or non-woven or a combination of films or layers which, in combination with the adhesive, show the desired characteristics of the patch. The film may for example be produced from a polyolefinic material, polyvinyl acetate (pVAc), polyester, polyamid, polyurethane material or polyethylene or copolymers or blends thereof. The backing layer may be vapour and water impermeable, or it may be water impermeable but vapour permeable.

### EXAMPLE

In order to treat callus caused by friction on the toes, such as corns, an adhesive patch comprising a polyurethane backing layer and a hydrocolloid adhesive layer is employed. An enzyme containing disc is placed on the skin-facing adhesive surface of the patch. The disc weighs about 10 mg and contains 10 to 20 mU of the enzyme and is placed centrally on the adhesive surface of the patch. Aside from the enzyme, the disc may comprise an inert carrier material that is solid at room temperature but liquefies at the temperature of the human skin. This may be cocoa butter or poly(ethylene glycol) of an appropriate molecular weight.

1. An adhesive patch for the removal or thinning of keratinaceous tissue, the patch comprising a first skin-facing surface and a second non-skin-facing surface, the patch comprises an adhesive layer, a backing layer and an enzyme material comprising a salmon zonase.

2. The patch according to claim 1, wherein the backing layer is located on the second surface of the patch.

3. The patch according to claim 1, wherein the adhesive layer is located on the first skin-facing surface of the patch.

4. The patch according to claim 1, wherein the enzyme material is located in one or more separate portions constituting at least a part of the skin-facing surface of the patch.

5. The patch according to claim 4, wherein the enzyme containing portion is sized to fit to a corn, wart or callous.

6. The patch according to claim 4, wherein the enzyme containing portion is embedded in the adhesive layer.

7. The patch according to claim 4, wherein enzyme containing portion is in the form of a layer overlying a part of the second surface of the adhesive layer.

8. The patch according to claim 4, wherein the enzyme material is incorporated in a pharmaceutically or cosmetically acceptable carrier.

9. The patch according to claim 4, wherein the carrier comprises poly(ethylene glycol).

10. The patch according to claim 4, wherein the carrier comprises cocoa butter.

11. The patch according to claim 1, wherein the backing layer is water impervious and vapour permeable.

12. A method for removing or thinning hyperkeratotic mammalian tissue comprising the steps of:
obtaining an adhesive patch comprising a backing layer covered on one side by an adhesive layer, the patch comprising enzyme material comprising a salmon zonase, and applying said patch to said hyperkeratotic tissue.

13. The method according to claim 12, comprising applying said patch to callous skin.

14. The method according to claim 12, comprising applying said patch to a corn.
